(19) United States
(12) Patent Application Publication
(10) Pub. No.: US 2020/0093971 A1
(43) Pub. Date: Mar. 26, 2020

(54) High Cycle Vacuum Breast Pump

(71) Applicant: Ameda, Inc., Buffalo Grove, IL (US)

(72) Inventors: Yordan Dimitrov Pargov, Rolling Meadows, IL (US); Kwang Suk Kim, Palatine, IL (US); David Shao Ling, Vernon Hills, IL (US)

(21) Appl. No.: 16/583,505

(22) Filed: Sep. 26, 2019

Related U.S. Application Data
(60) Provisional application No. 62/736,582, filed on Sep. 26, 2018.

(51) Int. Cl. A61M 1/06; A61M 1/00; A61M 39/22

(52) U.S. Cl. CPC A61M 1/06; A61M 39/223; A61M 1/0066

(57) ABSTRACT

A breast pump assembly is provided having a housing that is hermetically sealed to define a chamber within the housing, a pump disposed within the housing, wherein the pump is in fluid communication with the chamber and with an atmosphere outside the housing, and a valve system having a first, second, and third port, wherein the first port is in fluid communication with the atmosphere outside the housing, the second port is in fluid communication with the chamber within the housing, and the third port is configured to connect to a pumping kit such that the third port is in fluid communication with the pumping kit.

### CROSS REFERENCE TO RELATED APPLICATION

This application claims priority to U.S. Provisional Application No. 62/736,582, filed on Sep. 26, 2018, the content of which is hereby incorporated by reference.

### FIELD OF THE DISCLOSURE

The present disclosure generally relates to breast pumps and breast pump systems. More particularly, the present disclosure relates to a breast pump and breast pump system for extracting milk from a human breast in a quick and efficient manner.

### BACKGROUND OF THE INVENTION

Most prior art breast pumps are designed to extract milk from a user's breast(s) by cycling between applying a negative vacuum air pressure to the breast(s) and releasing the vacuum to simulate a baby's suckling. By altering between negative (or vacuum) pressure and then releasing the negative pressure, milk is expressed from the breast(s). Breast pumps generally cycle between these two states at a rate of 50 or more cycles per minute.

The power and efficacy of breast pumps is frequently defined by two variables: (1) the amount of cycles per minute achievable; and (2) the consistent strength of the vacuum during each cycle. Home use breast pumps are generally designed to be small and discrete so that a mother can carry the breast pump with her and use it throughout the day as necessary. However, these smaller portable pumps trade the power and efficacy of the system in favor of portability and size and thus are not able to reach desirable levels of cycles per minute nor a desirable vacuum strength. In contrast, hospital grade breast pumps, while powerful and effective enough to reach desirable levels of cycle rate and vacuum strength, are non-portable and thus not a viable option for a mother on the move. Thus, there is a need for a portable breast pump that has similar power and efficacy of hospital grade breast pumps.

In addition to lacking in power, prior art portable breast pumps are often noisy due to the noise created by continuously cycling between a negative vacuum pressure and then releasing the vacuum to atmospheric pressure. In addition, to cycle between these two states prior art breast pumps use a cycling motor as well, turning the motor on to create the vacuum and then off when the vacuum is released. These two features combine to create the undesirable cycling noise commonly associated with breast pumps. Users prefer a quiet system so that they can discretely use the breast pump at work and other public settings. Thus, there also exists a need for a portable breast pump that is quieter than prior art breast pumps.

### BRIEF SUMMARY OF THE INVENTION

In one aspect of the present disclosure, a breast pump assembly is provided. The breast pump assembly includes a housing, wherein the housing is hermetically sealed to define a chamber within the housing. The breast pump assembly also includes a pump disposed within the housing, wherein the pump is in fluid communication with the chamber and with an atmosphere outside the housing. The breast pump assembly further includes a valve system comprising a first port, a second port, and a third port, wherein the first port is in fluid communication with the atmosphere outside the housing, the second port is in fluid communication with the chamber within the housing, and the third port is configured to connect to a pumping kit such that the third port is in fluid communication with the pumping kit.

In other aspects of the present disclosure, the valve system may comprise a venting configuration and a vacuum configuration, wherein in the venting configuration, the valve system permits fluid flow between the first port and the third port, and in the vacuum configuration, the valve system permits fluid flow between the second port and the third port. In another aspect, in the venting configuration, the valve system may prevent fluid flow between the second port and the third port, and in the vacuum configuration, the valve system may prevent fluid flow between the first port and the third port. In still another aspect, the breast pump assembly may further include a control circuit board, wherein the control circuit board is configured to control operation of the valve system to move the valve system between the venting configuration and the vacuum configuration. In yet another aspect, the valve system may further include a solenoid, wherein when the solenoid is energized, the valve system moves from the venting configuration to the vacuum configuration. Further, the air in the chamber may be maintained at a pressure less than ambient air. In another aspect, the breast pump assembly may include at least one foam cushion disposed between the housing and at least one of the pump and valve system. In still another aspect, the breast pump assembly may also include a muffler and a fluid passageway, wherein the fluid passageway is in fluid communication with the muffler, the valve system, and the pump, wherein the pump is in fluid communication with the atmosphere outside the housing via the fluid passageway and the muffler, wherein the valve system is in fluid communication with the atmosphere outside the housing via the fluid passageway and the muffler.

In yet another aspect, the valve system may be a first valve system, the breast pump assembly further comprising a second valve system, wherein the second valve system comprises a fourth port, a fifth port, and a sixth port, wherein the fourth port is in fluid communication with the atmosphere outside the housing, the fifth port is in fluid communication with the chamber within the housing, and the sixth port is configured to connect to the pumping kit such that the sixth port is in fluid communication with the pumping kit. In yet another aspect, the pumping kit may be configured to engage with a user's breasts such that the third port of the first valve system is in fluid communication with one of the user's breasts and the sixth port of the second valve system is in fluid communication with the other of the user's breasts.

In another aspect of the present disclosure, a breast pump system is provided. The breast pump system includes an air-tight housing defining a storage chamber. The breast pump system also includes a pump disposed within the housing, the pump configured to extract air from the storage chamber to maintain a vacuum within the storage chamber. The breast pump system further includes a valve system comprising a first, second, and third port, wherein the first port is in fluid communication with an atmosphere outside the housing, the second port is in fluid communication with the storage chamber, and the third port is configured to be in fluid communication with a connectable pumping kit. In the system, the port system comprises a vacuum state and a venting state, wherein in the venting state, the valve system permits fluid flow between the first port and the third port, and the air within the valve system is at a pressure near atmospheric pressure, wherein in the vacuum state, the valve system permits fluid flow between the second port and the third port, and the valve system is at a pressure less than atmospheric pressure.

In another still further aspect of the present disclosure, in the breast pump system, in the venting state the valve system may be in fluid communication with the atmosphere outside the housing and not the air within the storage chamber, and in the vacuum state the valve system may be in fluid communication with the air within the storage chamber and not the atmosphere outside the housing. In yet another aspect, the breast pump system may also include a control circuit board, wherein the control circuit board is configured to move the valve system between the venting state and the vacuum state. The diaphragm may also include a muffler and a fluid passageway, wherein the fluid passageway is in fluid communication with the muffler, the valve system, and the pump, wherein the pump is in fluid communication with the atmosphere outside the housing via the fluid passageway and the muffler, wherein the valve system is in fluid communication with the atmosphere outside the housing via the fluid passageway and the muffler. In yet another aspect, the valve system may be normally in the venting state. Further, the valve system may also include a solenoid, wherein when the solenoid is energized it moves the valve system from the venting state to the vacuum state. In yet another aspect, the air in the storage chamber may be maintained at a pressure less than ambient air while the system is in the venting state and while the system is in the vacuum state.

In another aspect of the present disclosure, a method of controlling a breast pump system is provided. The method includes providing a breast pump system comprising an air-tight housing defining a storage chamber; a pump disposed within the housing, the pump configured to extract air from the storage chamber to maintain a vacuum within the storage chamber; and a valve system comprising a first, second, and third port, wherein the first port is in fluid communication with an atmosphere outside the housing, the second port is in fluid communication with the storage chamber, and the third port is configured to be in fluid communication with a connectable pumping kit. The method further includes activating the pump to extract air from the storage chamber and exhaust it to atmosphere. The method also includes moving the valve system to a vacuum state, wherein in the vacuum state the air within the valve system is in fluid communication with the air within the storage chamber and not in fluid communication with the atmosphere outside the housing, wherein in the vacuum state the air within the valve system is at a pressure less than atmospheric pressure. The method further includes moving the valve system to a venting state, wherein in the venting state the air within the valve system is in fluid communication with the atmosphere outside the housing and not in fluid communication with the air within the storage chamber, wherein in the venting state the air within the valve system is at a pressure near atmospheric pressure.

### BRIEF DESCRIPTION OF THE DRAWINGS

The disclosure will be more readily understood in view of the following description when accompanied by the below figures and wherein like reference numerals represent the elements, wherein:

FIG. 1 illustrates a schematic view of one embodiment of a breast pump system according to the present disclosure.

FIG. 2 illustrates an orthographic view of one embodiment of a breast pump system.

FIG. 3 illustrates a partial cross-section view of one embodiment of a breast pump system.

### DETAILED DESCRIPTION OF THE INVENTION

The following disclosure as a whole may be best understood by reference to the provided detailed description when read in conjunction with the accompanying drawings, drawing description, abstract, background, field of the disclosure, and associated headings. Identical reference numerals when found on different figures identify the same elements or a functionally equivalent element. The elements listed in the abstract are not referenced but nevertheless refer by association to the elements of the detailed description and associated disclosure.

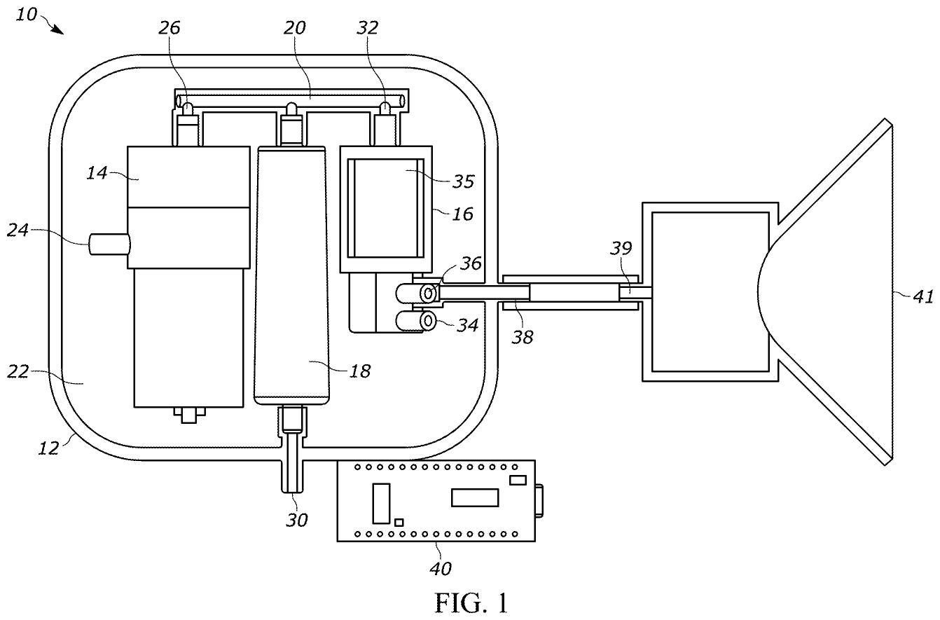

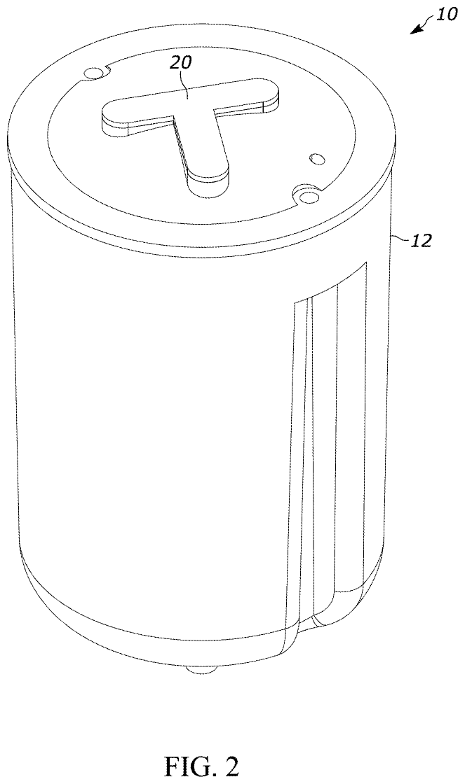

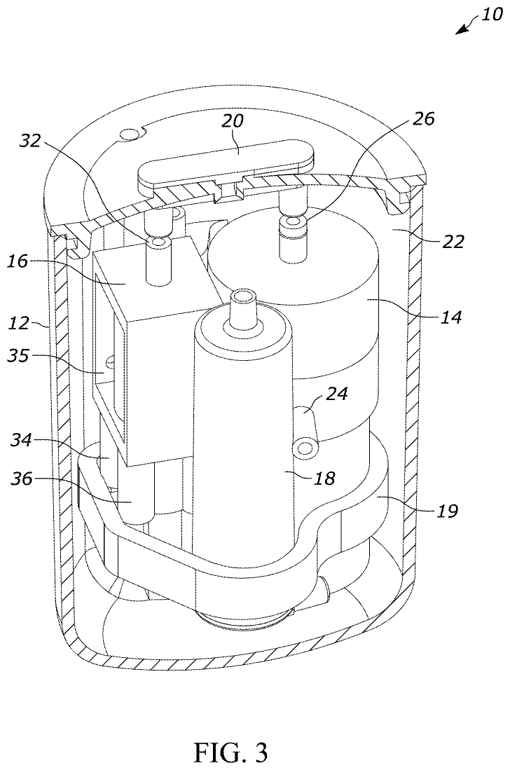

FIGS. 1, 2, and 3 show various view of an exemplary embodiment of a breast pump system 10. The breast pump system 10 may include a housing 12, within which may be disposed a pump 14, a three-way two-position valve 16, and a muffler 18. The pump 14, three-way valve 16, and muffler 18 may be connected via a passageway 20. In other embodiments, the pump 14, three-way two-position valve 16, and muffler 18 may be connected to each other via other methods or structures, or not connected at all as desired. The pump 14 may be any type of mechanical or electrical pump that is designed to extract air from a chamber to create a vacuum. One non-limiting example of an acceptable pump that may be used with this embodiment includes a rolling diaphragm pump, or aquarium pump. The muffler 18 may be any type of muffler used to reduce the noise of air travelling through it as known in the art. In this embodiment, the muffler 18 is a separate component from the housing 12, but in other embodiments the muffler 18 may be integrated directly into the housing 12.

The pump 14, three-way valve 16, and muffler 18 may be suspended within the housing 12 via foam padding 19 or other insulating and sound dampening material.

Alternatively, the pump 14, three-way valve 16, and muffler 18 may be connected to the housing 12 via other means known in the art. While this embodiment uses a muffler 18, in alternative embodiments the muffler 18 may be omitted entirely.

The housing 12 may be hermetically sealed such that air cannot travel through the housing 12, thereby creating an air-tight chamber 22 within the housing 12. When the breast pump system 10 is in operation, the chamber 22 is ideally maintained at a pressure less than the pressure of ambient air outside of the chamber 22, thereby creating a vacuum. To maintain the chamber 22 as a vacuum, the pump 14, via port 24, pumps air out of the chamber 22 and expels it to atmosphere. In this embodiment, the air extracted by the pump 14 is exhausted from the pump 14 through a port 26 into the passageway 20 and then into the muffler 18 and then out through an exhaust port 30 into the ambient air. However, the air extracted by the pump 14 can alternatively be exhausted directly into the atmosphere from the pump 14 or through other means.

The three-way valve 16 may include a first port, or vent port, 32 that is in fluid communication with the passageway 20, a second port, or vacuum port, 34 that is in fluid communication with the chamber 22, and a third port, or common/kit port, 36 that is in fluid communication with an outlet port 38. The three-way two-position valve 16 may allow fluid flow in two directions, either between the chamber 22 and the outlet port 38 or between the outlet port 38 and the passageway 20. The three-way valve 16 may also include a small chamber or passageway 35 which permits fluid communication between the ports 32, 34, and 36. The outlet port 38 may be connected to a pumping kit, or breast interface, 41 via a tube 39 which engages a user's breast(s). The ports 32, 34, and 36 may be controlled by one or more solenoids (not shown) that may be energized and de-energized to open and close the ports. The ports 32, 34, 36 may alternatively be controlled by any other means, including mechanical and electrical control systems known in the art. In this embodiment, the three-way valve 16 is controlled by a single solenoid with two valve configurations allowing fluid flow in two directions, either between the third port 36 and the first port 32 or between the third port 36 and the second port 34. The first valve configuration, also known as the venting configuration, permits fluid flow between the first port 32 and the third port 36. The second valve configuration, also known as the vacuum configuration, permits fluid flow between the third port 36 and the second port 34. In this embodiment, the three-way valve 16 is normally in the venting configuration when the solenoid is de-energized and the three-way valve 16 moves to the vacuum configuration when the solenoid is energized. However, the three-way valve 16 may also be modified such that the three-way valve 16 is normally in the vacuum configuration when the solenoid is de-energized and the three-way valve 16 moves to the venting configuration when the solenoid is energized. While this embodiment describes the use of a three-way valve 16, any valve system that is capable of controlling fluid flow between the first, second, and third ports as described above may be used, including but not limited to four-way and five-way valves.

A control circuit board 40 may be attached to the housing 12 or otherwise connected to the breast pump system 10 to control the operation of the breast pump system 10. The control circuit board 40 may control the power and on/off function of the pump 14, the movement of the three-way valve 16 between the venting and vacuum configurations, and any other components of the breast pump system 10 as necessary. It may also control the cycle rate of the system 10.

In this embodiment, the pump 14, three-way valve 16, and muffler 18 are all disposed within the chamber 22. While this is the preferred embodiment so that the breast pump system 10 has a smaller profile, one or all of the pump 14, three-way valve 16, and muffler 18 may be disposed outside of the chamber 22. The size of the chamber 22 may be varied as desired so long as there is a sufficient volume to maintain a vacuum within it throughout the entire or most of the operation of the breast pump system 10. Further, while the present embodiment utilizes a cylindrical housing 12, the housing 12 (as well as all other components) may be different shapes as desired. In addition, while the present embodiment uses a passageway 20 to facilitate communication between the pump 14, three-way valve 16, and muffler 18, the passageway 20 may be modified or omitted entirely as desired. For example, in one alternative embodiment, the passageway 20 and muffler 18 may be omitted from the design and the first port 32 of the three-way valve 16 may be in direct communication with the atmosphere while the port 26 of the pump 14 is also in direct communication with the atmosphere.

In use, the breast pump system 10 is controlled to a create a drive cycle that alternates between a negative pressure or vacuum state and a positive pressure or venting state. Essentially, the breast pump system 10 first applies a negative vacuum pressure to the user's breast(s) and then opens a port in communication with the atmosphere to return the negative vacuum pressure back to or near atmospheric pressure. The system 10 repeats this cycle indefinitely to simulate a baby's suckling. During use, the pump 14 remains on at all times to continuously remove any excess air from the chamber 22, thereby maintaining a vacuum within the chamber 22.

While the pump 14 continuously pumps air out of the chamber 22, the three-way valve 16 is periodically moved between the venting configuration and the vacuum configuration via the control circuit board 40 to create the above-mentioned pressure cycle. A single cycle of operation occurs as follows. The cycle begins with the solenoid that controls the three-way valve 16 in a de-energized state, thus leaving the three-way valve in its natural, venting configuration. Because the three-way valve 16 is in the venting configuration, fluid flow is permitted between the first port 32 and the third port 36, thus allowing fluid communication between the passageway 20, the muffler 18, and the pumping kit 41 which is in contact with the user's breast(s). Thus, because all of these portions of the system 10 are in fluid communication with the outside atmosphere, the air contained within them, including the air within the three-way valve 16 and the pumping kit 41, is at or nearly at atmospheric pressure.

To begin the cycle, the control circuit board 40 directs the solenoid to energize, thereby causing three-way valve 16 to move to the vacuum configuration, which cuts off the three-way valve 16 and the pumping kit 41 from fluid communication with the outside atmosphere. Because the three-way valve is now in the vacuum configuration, fluid flow is permitted between the second port 34 and the third port 36, thus allowing fluid communication between the vacuum within the chamber 22 and the pumping kit 41 which is in contact with the user's breast(s). Thus, while communication with the atmosphere is closed, the pressure within the three-way valve 16 and the pumping kit 41 in contact with the user's breast(s) quickly plummets as the combined volume of the pumping kit 41, chamber 22, and three-way valve 16 naturally reaches an equilibrium pressure that is less than atmospheric pressure. This quick drop in pressure applied to the user's breast(s) simulates a baby's suckling and thus causes milk to be excreted from the user's breast(s). In some embodiments, the pumping kit 41 may be connected to or also include a milk collection chamber that collects any milk that is excreted from the user's breast(s).

To complete the cycle, the solenoid controlling the three-way valve 16 is de-energized. This action results in movement of the three-way valve 16 from the vacuum configuration back to the venting configuration, thereby closing off fluid communication between the chamber 22 and the three-way valve 16 and pumping kit 41. At this point, the chamber 22 is ideally still at a pressure less than atmospheric pressure because the volume of the chamber 22 is preferably greater than the volume of the three-way valve 16 and pumping kit 41. Thus, a vacuum remains in the chamber 22 and the pump 14 continues to run to strengthen the vacuum within the chamber 22. This reservoir concept allows a vacuum to be stored and ready for use throughout the entire operation of the breast pump system 10.

In addition to the effect on the chamber 22 described above, when the solenoid described above is de-energized and the three-way valve 16 returns to the venting configuration, the three-way valve 16 and pumping kit 41 reestablish fluid communication with the atmosphere, and thus the air within, which is currently at a negative pressure, is quickly returned to atmospheric pressure as ambient air rushes into the three-way valve 16 from the passageway 20 (and the muffler 18 and port 30) and through the first port 32. This quick change in pressure once again is applied to the user's breast(s) and simulates a baby's suckling.

Once the air within the three-way valve 16 and pumping kit 41 are returned to atmospheric pressure (or close to atmospheric pressure), the cycle begins again with the control circuit board 40 activating the solenoid to move the three-way valve 16 back to the vacuum configuration.

While the present embodiment utilizes a specific solenoid powered three-way valve 16, other valve systems may be used as desired. For example, the three-way valve 16 may use individually controlled ports such that the first and second ports 32, 34 may be opened and closed independently of the other. This may be desirable to ensure that the vacuum stored in the chamber 22 is not unnecessarily vented to atmosphere during the second part of the drive cycle. To achieve this the second port 32 may be closed first and then the first port 34 is subsequently opened. Other variations are possible as desired.

While the present embodiment utilizes a single pump 14, a single chamber 22, and a single three-way valve 16, the disclosure is not so limited. For example, it may be desirable to apply varying pressures or cycles to the user's individual breasts. As such, the present embodiment may also utilize a second pump, a second chamber, and a second three-way valve, or any variation thereof. In one non-limiting example, a second three-way valve may be used that is connected to the chamber 22. The second three-way valve may be connected to one of the user's breasts while the first three-way valve 16 may be connected to the other of the user's breasts. In this way, the first and second three-way valves may be individually controlled to effect a vacuum cycle on each individual breast. In another non-limiting example, a second three-way valve, second pump, and second chamber may be used. The second three-way valve, second pump, and second chamber may operate independently of the first three-way valve 16, first pump 14, and first chamber 22. In this way one half of the system may control the vacuum cycle for one of the user's breasts while the other half of the system may control the vacuum cycle for the other of the user's breasts.

The breast pump system 10 described herein has multiple advantages over prior art breast pumps. First, because the breast pump system 10 includes a vacuum storage chamber such as the chamber 22 described above. Prior art portable breast pumps do not contain vacuum storage chambers. Rather, they rely on the pump to create a new vacuum from atmospheric pressure every single cycle. Because the motors are generally small for portability's sake, the pump cannot quickly create an effective vacuum, and this lowers either the strength of the vacuum during the vacuum cycle or lowers the cycle rate. Studies show that a baby suckles at rates of up to 120 cycles per minute. This rate is unachievable with prior art designs. However, because the present invention utilizes a vacuum storage chamber, the cycle rate can be increased significantly over prior art designs without sacrificing portability and vacuum strength. For example, testing has shown that the present invention can achieve a vacuum level of −250 mmHg at 55 cycles per minute or a maximum cycle rate of 120 cycles per minute at a vacuum strength of −40 to −170 mmHg. These two exemplary rates and accompanying vacuum strengths are unachievable by current portable breast pumps on the market. However, the cycle rates and vacuum strengths are not limited to these two examples and may be varied as desired, including a cycle rate of between 20 and 140 cycles per minute and a vacuum level of between −40 and −250 mmHg.

The vacuum chamber 22 of the breast pump system 10 provides additional advantages to the present invention. Because the chamber 22 doubles as a housing 12 for the other components of the system 10, the profile of the system 10 is significantly reduced and thus more desirable as a portable breast pump system 10. In addition, because the components are suspended within the chamber 22 via foam padding 19, the noise created by the components during operation is significantly reduced. First, the foam padding 19 helps dampen any vibrations and thus reduces the noise. Second, because the chamber 22 is maintained at a negative pressure, sound waves do not transfer through the vacuum as readily as they do through atmosphere, thereby further reducing the noise created by the operation of the system 10.

While the disclosure herein is focused on the use of the invention with a breast pump, the disclosure is not so limited. Rather, this invention can be used with any system or process that requires an alternating vacuum system, including but not limited to wound therapy systems.

The above detailed description and the examples described therein have been presented for the purposes of illustration and description only and not by limitation. It is therefore contemplated that the present disclosure cover any and all modifications, variations or equivalents that fall within the spirit and scope of the basic underlying principles disclosed above and claimed herein.

1. A breast pump assembly comprising:
a housing, wherein the housing is hermetically sealed to define a chamber within the housing;
a pump disposed within the housing, wherein the pump is in fluid communication with the chamber and with an atmosphere outside the housing;
a valve system comprising a first port, a second port, and a third port, wherein the first port is in fluid communication with the atmosphere outside the housing, the second port is in fluid communication with the chamber within the housing, and the third port is configured to connect to a pumping kit such that the third port is in fluid communication with the pumping kit.

2. The breast pump assembly of claim 1, wherein the valve system comprises a venting configuration and a vacuum configuration, wherein in the venting configuration, the valve system permits fluid flow between the first port and the third port, and in the vacuum configuration, the valve system permits fluid flow between the second port and the third port.

3. The breast pump assembly of claim 2, wherein in the venting configuration, the valve system prevents fluid flow between the second port and the third port, and in the vacuum configuration, the valve system prevents fluid flow between the first port and the third port.

4. The breast pump assembly of claim 3, further comprising a control circuit board, wherein the control circuit board is configured to control operation of the valve system to move the valve system between the venting configuration and the vacuum configuration.

5. The breast pump assembly of claim 4, wherein the valve system is normally in the venting configuration.

6. The breast pump assembly of claim 5, wherein the valve system further comprises a solenoid, wherein when the solenoid is energized, the valve system moves from the venting configuration to the vacuum configuration.

7. The breast pump assembly of claim 1, wherein the air in the chamber is maintained at a pressure less than ambient air.

8. The breast pump assembly of claim 1, further comprising at least one foam cushion disposed between the housing and at least one of the pump and valve system.

9. The breast pump assembly of claim 1, further comprising a muffler and a fluid passageway, wherein the fluid passageway is in fluid communication with the muffler, the valve system, and the pump, wherein the pump is in fluid communication with the atmosphere outside the housing via the fluid passageway and the muffler, wherein the valve system is in fluid communication with the atmosphere outside the housing via the fluid passageway and the muffler.

10. The breast pump assembly of claim 1, wherein the valve system is a first valve system, the breast pump assembly further comprising a second valve system, wherein the second valve system comprises a fourth port, a fifth port, and a sixth port, wherein the fourth port is in fluid communication with the atmosphere outside the housing, the fifth port is in fluid communication with the chamber within the housing, and the sixth port is configured to connect to the pumping kit such that the sixth port is in fluid communication with the pumping kit.

11. The breast pump assembly of claim 10, wherein the pumping kit is configured to engage with a user's breasts such that the third port of the first valve system is in fluid communication with one of the user's breasts and the sixth port of the second valve system is in fluid communication with the other of the user's breasts.

12. The breast pump assembly of claim 1, wherein the valve system is a first valve system, the pump is a first pump, and the chamber is a first chamber, the breast pump assembly further comprising a second valve system, a second pump, and a second chamber, wherein the second pump is disposed within the housing and is in fluid communication with the second chamber and with the atmosphere outside the housing, wherein the second valve system comprises a fourth port, a fifth port, and a sixth port, wherein the fourth port is in fluid communication with the atmosphere outside the housing, the fifth port is in fluid communication with the second chamber within the housing, and the sixth port is configured to connect to the pumping kit such that the sixth port is in fluid communication with the pumping kit.

13. A breast pump system comprising:
an air-tight housing defining a storage chamber;
a pump disposed within the housing, the pump configured to extract air from the storage chamber to create a vacuum within the storage chamber; and
a valve system comprising a first, second, and third port, wherein the first port is in fluid communication with an atmosphere outside the housing, the second port is in fluid communication with the storage chamber, and the third port is configured to be in fluid communication with a connectable pumping kit;
wherein the valve system comprises a vacuum state and a venting state, wherein in the venting state, the valve system permits fluid flow between the first port and the third port, and the air within the valve system is at a pressure near atmospheric pressure, wherein in the vacuum state, the valve system permits fluid flow between the second port and the third port, and the valve system is at a pressure less than atmospheric pressure.

14. The breast pump system of claim 13, wherein in the venting state the valve system is in fluid communication with the atmosphere outside the housing and not the air within the storage chamber, and in the vacuum state the valve system is in fluid communication with the air within the storage chamber and not the atmosphere outside the housing.

15. The breast pump system of claim 13, further comprising a muffler and a fluid passageway, wherein the fluid passageway is in fluid communication with the muffler, the valve system, and the pump, wherein the pump is in fluid communication with the atmosphere outside the housing via the fluid passageway and the muffler, wherein the valve system is in fluid communication with the atmosphere outside the housing via the fluid passageway and the muffler.

16. The breast pump system of claim 13, further comprising a control circuit board, wherein the control circuit board is configured to move the valve system between the venting state and the vacuum state.

17. The breast pump system of claim 13, wherein the valve system is normally in the venting state.

18. The breast pump system of claim 17, wherein the valve system further comprises a solenoid, wherein when the solenoid is energized it moves the valve system from the venting state to the vacuum state.

19. The breast pump assembly of claim 13, wherein the air in the storage chamber is maintained at a pressure less than ambient air while the system is in the venting state and while the system is in the vacuum state.

20. A method of controlling a breast pump system comprising:
providing a breast pump system comprising an air-tight housing defining a storage chamber; a pump disposed within the housing, the pump configured to extract air from the storage chamber to maintain a vacuum within the storage chamber; and a valve system comprising a first, second, and third port, wherein the first port is in fluid communication with an atmosphere outside the housing, the second port is in fluid communication with the storage chamber, and the third port is configured to be in fluid communication with a connectable pumping kit;
activating the pump to extract air from the storage chamber and exhaust it to atmosphere;
moving the valve system to a vacuum state, wherein in the vacuum state the air within the valve system is in fluid communication with the air within the storage chamber and not in fluid communication with the atmosphere outside the housing, wherein in the vacuum state the air within the valve system is at a pressure less than atmospheric pressure; and
moving the valve system to a venting state, wherein in the venting state the air within the valve system is in fluid communication with the atmosphere outside the housing and not in fluid communication with the air within the storage chamber, wherein in the venting state the air within the valve system is at a pressure near atmospheric pressure.
